(19) United States
(12) Patent Application Publication
(10) Pub. No.: US 2012/0165623 A1
(43) Pub. Date: Jun. 28, 2012

(54) Relational Thermorespirometer Spot Vitals Monitor

(76) Inventors: Lawrence Allan Lynn, Columbus, OH (US); Christopher Noel Cindrich, Draper, UT (US)

(21) Appl. No.: 13/392,827

(22) PCT Filed: Aug. 27, 2010
(86) PCT No.: PCT/GB10/01624

§ 371 Date: Feb. 27, 2012

Related U.S. Application Data
(60) Provisional application No. 61/275,384, filed on Aug. 28, 2009.
(60) Provisional application No. 61/340,108, filed on Mar. 12, 2010.

(51) Int. Cl. A61B 5/0205; A61B 5/1455; A61B 5/087; A61B 5/01; A61B 5/08

(52) U.S. Cl. 600/301

(57) ABSTRACT

A vitals monitor [10] includes an elongated probe [20] having a distal tip [28], the monitor having a distal sensor surface [24] adjacent the distal tip for sensing oral temperature of a patient and at least one proximal gas receiver [30] positioned along the probe [20] so that, when the distal tip [28] is placed under a patients tongue, exhaled gas from the nose strikes the gas receiver [30] for detecting and/or measuring at least one signal indicative of at least one ventilation parameter.

### RELATED APPLICATIONS

Application Ser. No. 11/351,449 filed Feb. 10, 2006, currently pending, is incorporated by reference as if completely disclosed herein.

### BACKGROUND

Embodiments of the present disclosure may relate to a system and method for evaluating physiological parameter data. In embodiments, a medical device may be capable of determining the respiratory rate and other parameters. In one embodiment this is achieved using a digital thermometer interface.

This section is intended to introduce the reader to various aspects of art that may be related to various aspects of the present disclosure, which are described and/or claimed below. This discussion is believed to be helpful in providing the reader with information to facilitate a better understanding of the various aspects of the present disclosure. Accordingly, it should be understood that these statements are to be read in this light, and not as admissions of prior art.

Spot vitals are an important part of patient management. Despite the importance of ventilation parameters in the assessment of patients, ventilation parameters are often determined as part of the spot vitals in a subjective manner, as for example by manually counting tidal breaths. Such subjective determinations are often unreliable and are often very poor indicators of tidal amplitude and of the timed changes in tidal amplitude and other ventilation parameters. While the photoplethysmographic time series, as for example derived from a pulse oximeter, can be used to determine the ventilation parameters such as respiratory rate, as discussed for example in U.S. patent application Ser. No. 11/708,422, the contents of which are incorporated by reference as if completely disclosed herein, this signal is affected by blood volume, blood perfusion, vascular factors, as well as respiratory effort.

Automated systems for objectively measuring oral temperature are in wide use. Conventional oral thermometers comprise a digital elongated thermometer with a distal temperature sensor adjacent the tip for insertion into the sublingual pocket. The elongated thermometer is often comprised of a pole which is often comprised of metal which, during use, is encased in a disposable, single use plastic housing which covers the elongated thermometer to prevent transmission of microorganisms between patients.

### SUMMARY OF THE INVENTION

The present invention has been devised in order to address one or more drawbacks of known systems. Preferably, the present invention avoids, ameliorates or even overcomes one or more such drawbacks. Certain preferred aspects of preferred embodiments of this disclosure are set forth below. It should be understood that these aspects are presented merely to provide the reader with a brief summary of certain forms the disclosure might take and that these aspects are not intended to limit the scope of the disclosure. Indeed, the disclosure may encompass a variety of aspects that may not be set forth below. Unless the context demands otherwise, features of any aspect or embodiment may be combined with each other, singly or in any combination.

Preferably, a spot or continuous vitals signs monitor is provided which simultaneously detects at least one respiratory parameter and a body temperature which may be a sublingual temperature. Preferred embodiments may include an oral thermometer further comprising at least one gas receiving sensor for detecting and/or measuring at least one ventilation parameter. The ventilation parameter can be indicative of a flow and or volume of at least one exhaled and/or inhaled gas. In some preferred embodiments the sensor can be proximal to the distal tip. At least one sensor can be positioned along an elongated oral thermometer so that the sensor is proximal to the tongue when the thermometer tip is positioned in the sublingual pocket so that the sensor can be in contact with ventilated gas moving through the mouth and/or nose. The sensor further can be positioned so that it is proximal to the lips when the thermometer tip is positioned in the sublingual pocket so that the sensor can be in contact with ventilated gas moving through the nose. This allows the body temperature and parameters indicative of ventilation, such as a time series of temperature, gas pressure or percentage thereof; the respiration rate, the inspiration time, the inspiration amplitude, the inspiration slope, the expiration time, expiration amplitude, expiration slope, and/or another primary or relational wave component and/or derivative (as for example the area under the curve) to be measured simultaneously or otherwise, with the same interface. The pattern of ventilation can also be detected, as for example the pattern of Cheyne-Stokes respiration, bradypnea, and/or Biots respiration to name a few. The components and partial pressures of the exhaled gas can be detected, as for example by a high resolution time-series (up to 256 Hz or more) of the partial pressure of CO₂ and the waveform components and or derivative of this gas component can be identified, as for example those mentioned above and combined with another measure such as nasal pressure or nasal gas temperature. The components of the time-series of the exhaled CO₂ and nasal pressure, nasal gas temperature, and or another measure such as exhaled nitric oxide, methyl nitrate, hydrogen peroxide, carbon monoxide, breath condensate, ethanol or other small molecules, to name a few, may all be derived and compared to other parameters such as the SPO₂.

Preferably, at least one gas receiving sensor is positioned along an elongated oral thermometer so that, in use, exhaled gas from the nose strikes the sensor to generate a signal indicative of at least one ventilation parameter. The gas receiving sensor can be a thermistor such as a rapid response thermistor for sensing temperature change indicative of the times-series of tidal breaths. The sensor can be a CO₂ sensor, as for example a mainstream or side-stream CO₂ sensor, a carbon nanotube sensor, a fiber-optic sensor, an electromagnetic sensor, an infra-red sensor, a pressure sensor, a flow sensor, a pH sensor, a piezoelectric sensor, a chemiresistor array, laser spectrometer, a PVC or other pressure and or heat sensitive film sensor, a gas collector such as negative pressure tube and/or collector which faces the nasal gas stream and which carries the exhaled gas to a remote sensor, to name a few.

Preferably, there is additionally provided a pulse oximeter which generates a photoplethysmographic output and or a derived output from the photoplethysmographic output, as for example a respiratory rate and respiratory pattern derived from that output. The photoplethysmographic output and/or the derived output is/are compared with output of the gas receiving sensor and/or derived output(s) from the gas receiving sensor for example, the respiratory rate and pattern derived from the gas receiving sensor. Since artifacts may affect either output, the displayed output may include values derived from both sources, a combination value derived from both sources and the most reliable value, as for example the value with the highest signal to noise and/or the least intruding artifact. The outputs from both sources can be compared over time, with periodic check of the spot signal from the gas receiving sensor being used to confirm and/or calibrate a continuous respiratory rate calculation from the photoplethysmographic output.

Preferably, the signals are integrated and an index of ventilation instability and/or hemodynamic instability is provided. For example, a ventilation instability index may be provided. This may be bi-phasic providing a positive or negative number depending on the respiration rate (RR) above or below normal and further depending on the SPO₂ in relation to the associated respiratory rate. This protects the user against a false sense of security which may be provided by a normal SPO₂ in the presence of hyperventilation. For example, in response to an SPO₂ of 92 in the presence of a respiratory rate of 12 the processor may be programmed not to generate any warning and may, for example generate a ventilation oximetry index (VOI) of 0. In the alternative the same SPO₂ of 92 when associated with a RR of 24 may generate a warning “SPO₂ low in relation to high RR” and/or may produce a VOI value of +3 indicative of a relationally abnormal SPO₂ and a high RR. In this way both sensitivity and specificity for instability are enhanced.

Preferably, an exhalation sensor, such as an infrared sensor which may include an automatic distance indicator to assure the distance is equal each time the measurement is made, is provided, as for example a handheld unit which can comprise a fast response infrared camera. The processor may be programmed to count the respiration rate and derive the ventilation time series and determine the other parameters both individually and relationally as discussed. Preferred embodiments including this feature the advantage of avoiding a proximal heat sensor in the thermometer but positioning may be more expensive than the aforementioned embodiments.

### BRIEF DESCRIPTION OF THE DRAWINGS

FIG. 1 is an elevation view of a thermorespirometer and an accompanying processor wherein the two are connected for direct data transmission from the thermorespirometer to the processor;

FIG. 2 is an elevation view of a thermorespirometer probe and an accompanying processor configured for wireless transmission of data from the thermorespirometer to the processor;

FIG. 3 is an elevation view of a disposable sheath;

FIG. 4 is an elevation view of a thermorespirometer probe of the present invention;

FIG. 5 is an elevation view of a thermorespirometer probe with the disposable sheath in place;

FIG. 6 is an end view of a thermorespirometer probe;

FIG. 7 is an end view of a thermorespirometer probe illustrating flow of gas across the surface of the probe;

FIG. 8 is a section view of typical human oral and nasal cavities with a thermorespirometer probe shown in place;

FIG. 9 is an elevation view of an alternative embodiment of a thermorespirometer probe;

FIG. 10 is a side elevation view of an alternative embodiment of a thermorespirometer probe;

FIG. 11 is an isometric view of an alternative embodiment of a thermorespirometer probe;

FIG. 12 is a side elevation view of an alternative embodiment of a disposable sheath;

FIG. 13 is a side elevation view of an alternative embodiment of a thermorespirometer probe with the disposable sheath in place;

FIG. 14 is an end view of an alternative embodiment of a thermorespirometer probe illustrating flow of gas across the surface of the probe;

FIG. 15 is a section view of typical human oral and nasal cavities with an alternative embodiment of a thermorespirometer probe shown in place;

FIG. 16 is an elevation view of a thermorespirometer and an accompanying processor wherein a multitude of physiologic parameters can be acquired;

FIG. 17 is an isometric view of an alternative embodiment of a thermorespirometer probe;

FIG. 18 is an isometric view of an alternative embodiment of a thermorespirometer probe.

### DETAILED DESCRIPTION OF SPECIFIC EMBODIMENTS, FURTHER OPTIONAL FEATURES OF THE INVENTION

Embodiments will be described below. In an effort to provide a concise description of these embodiments, not all features of an actual implementation are described in the specification. It should be appreciated that in the development of any such actual implementation, as in any engineering or design project, numerous implementation specific decisions must be made to achieve the developers' specific goals, such as compliance with system-related and business-related constraints, which may vary from one implementation to another. Moreover, it should be appreciated that such a development effort might be complex and/or time consuming, but would nevertheless be a routine undertaking of design, fabrication, and manufacture for those of ordinary skill having the benefit of this disclosure.

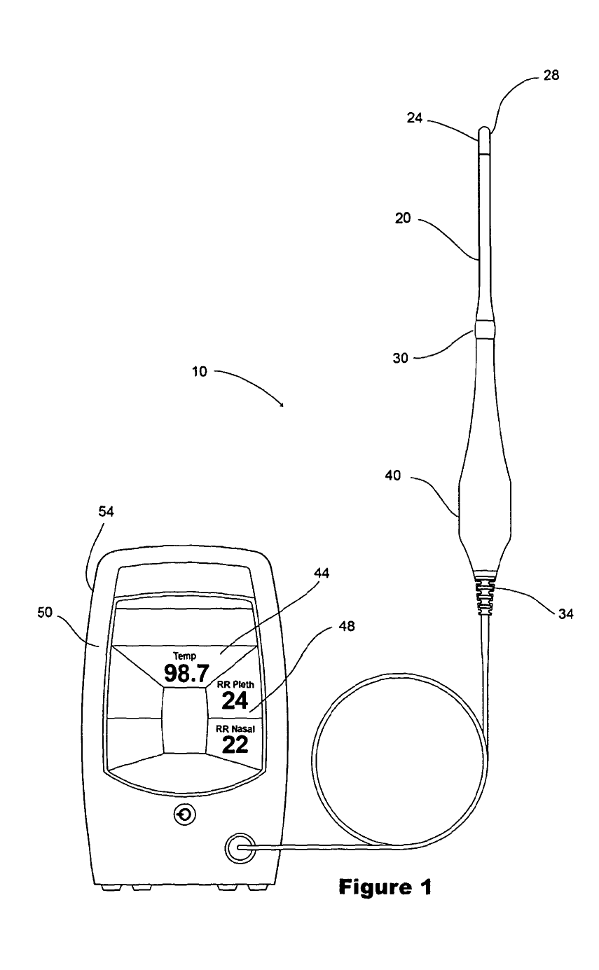

As shown in FIG. 1, one embodiment of the vitals monitor 10 includes an elongated pole 20 having a distal sensor surface 24 adjacent the distal tip 28 of the pole 20 for insertion into the sublingual pocket of a patient, a proximal sensor surface 30 is mounted along the pole 20 in a position such that the proximal sensor surface 30 is proximal the lips of the patient when the distal sensor surface 24 is positioned within the sublingual pocket. In one example for adult use, the sensor surface 30 is positioned 5 cm from the tip and may extend or be positioned along a range of 3-12 cm from the tip. The position can be shortened for pediatric use or the sensor surface 30 may be elongated (or multiple sensors deployed along the pole) to accommodate a range of positions. In this embodiment the pole 20 contains a distal connector 34 (such as a wire or tube) connected to one or more sensor surfaces in or on pole 20. Pole 20 and sensor surfaces 24 and 30 may be comprised of the same materials or dissimilar materials. Further, pole 20 and sensor surfaces 24 and 30 may be comprised of plastic, metal, or a combination of both. Pole 20 may be one diameter, multiple diameters or a combination thereof as desirable to facilitate function or manufacturing of the device. The pole 20 is connected to a hub 40 which is sized to be held by the fingers of a user. The distal connector 34 extends from the hub 40 to connect to a body temperature processor 44 and a respiration processor 48 which can be combined with or part of a main processor 50 in a separate casing 54.

FIG. 2 shows an alternative design where the hub 40 can contain a primary processor 41 which can include an analog to digital converter and a wireless transmitter which transmits a secondary processor so that the hub 40 and pole 20 are free from wired connection to a separate casing.

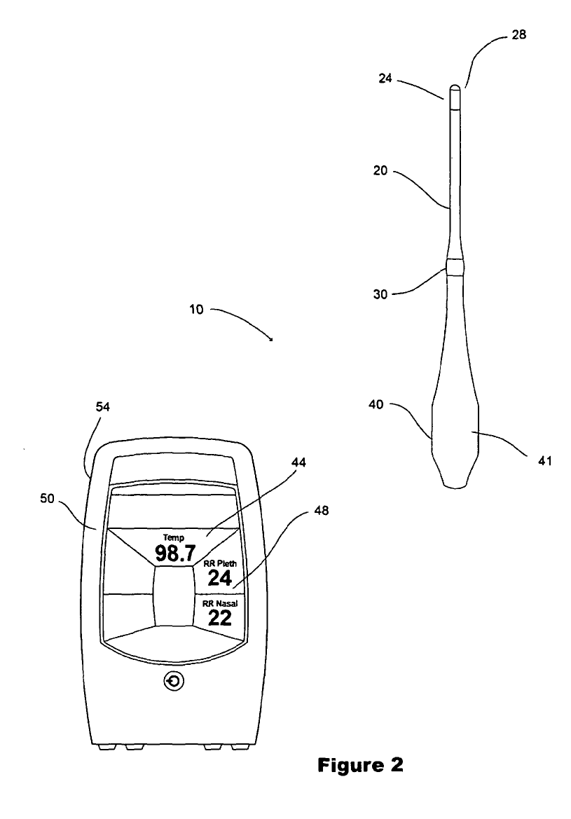

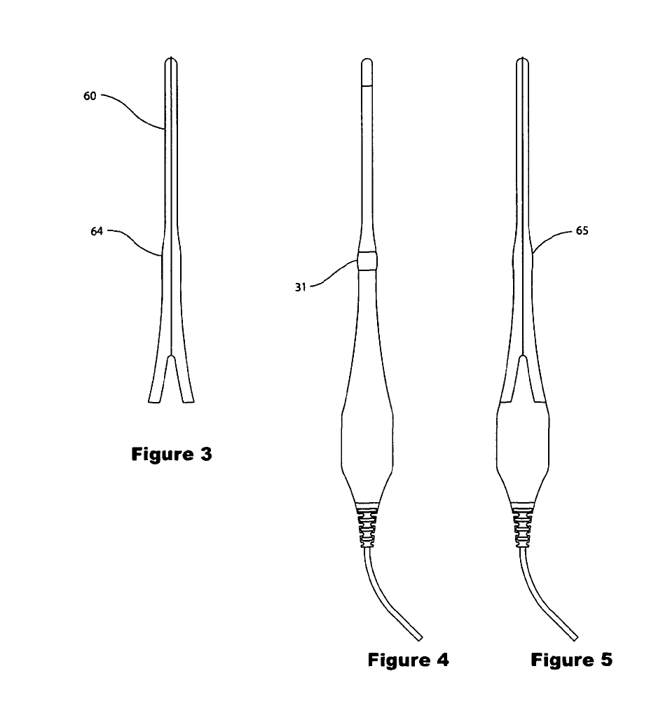

FIG. 3 discloses a flexible plastic cover 60 which is provided for use in covering all or portions of an elongate pole as described in FIG. 1. Cover 60 can be comprised of a plastic which readily transmits heat energy as is known in the art. Cover 60 can be of uniform thickness or have a thin region 64 for positioning adjacent a proximal sensor (not shown) which can be stretched by the sensor so that heat energy is more rapidly transmitted to the sensor. Further, cover 60 can have a shape or profile substantially equal to the shape or profile of the elongate pole (not shown) such that sliding placement of cover 60 on the elongate pole will be substantially uninhibited except for any surfaces or features of the elongate pole which are generally anomalous to the overall contour of the elongate pole which will induce deformation of cover 60 resulting in stretching of cover 60 local to that feature. Alternatively a tube for transmitting nasal pressure (not shown) and/or to receive gas transmitted through the tube as for example by negative pressure applied through the tube may be mounted with or comprise an integral part of plastic housing 60 with a gas collection or exit point at one or more locations along plastic housing 60 to receive pressure waveforms and/or gas derived from nasal or oral breathing.

FIG. 4 shows sensor surface 30 having a bulge 31 designed to stretch a cover (not shown) such that there is enhanced contact of the cover to sensor surface 30. Bulge 31 could be varied in size and shape in order to ensure effective stretching and enhanced contact of the cover (not shown) to bulge 31.

FIG. 5 shows cover 60 in place over pole 20 with region 64 being stretched by the bulge of a sensor surface beneath cover 60 such that contact 65 is made between cover 60 and the bulging sensor surface for rapid and effective transmission of heat energy across one or more material layers.

FIG. 6 shows cylindrical nature of pole 20, sensor surface 30 and hub 40 which facilitate use of the device with no orientation required toward specific directional heat sources.

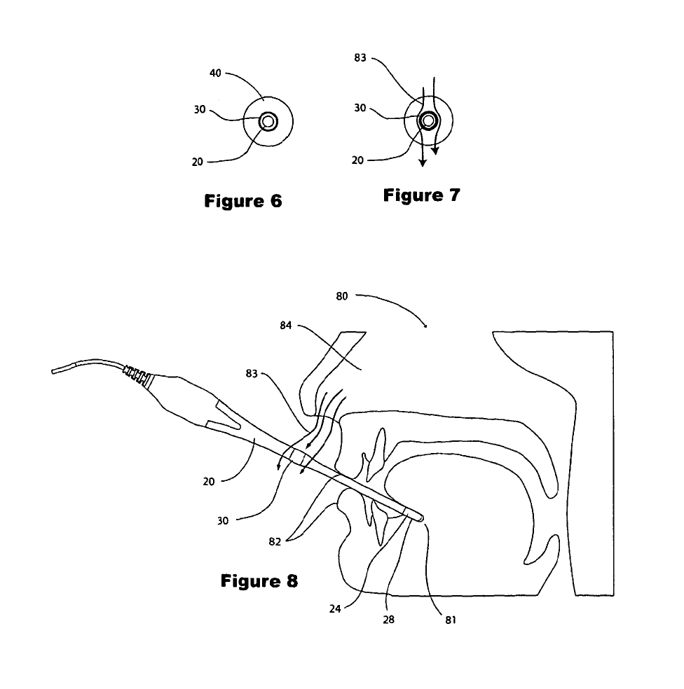

FIG. 7 illustrates directional flow of gas 83 across sensor surface 30 of pole 20. Sensor surface 30 could house, cover or otherwise contain one or more discreet physical parameter sensors such as a thermistor. The sensors can be arrayed with equal spacing between them or may be clustered in non-uniform groups. Use of 2 or more sensors within or beneath sensor surface 30 would promote greater sensitivity of the sensor cluster despite relative orientation of sensor surface 30 to a directional heat source.

FIG. 8 is a cutaway view of the oral and nasal cavity 80 of a human being with pole 20 shown in place. Sensor surface 24 of distal tip 28 is shown in the sublingual pocket 81. The lips 82 are shown pressed to pole 20 in order to affect a seal on pole 20 ensuring exhaled gas 83 flows from the nasal cavity 84 onto and across sensor surface 30.

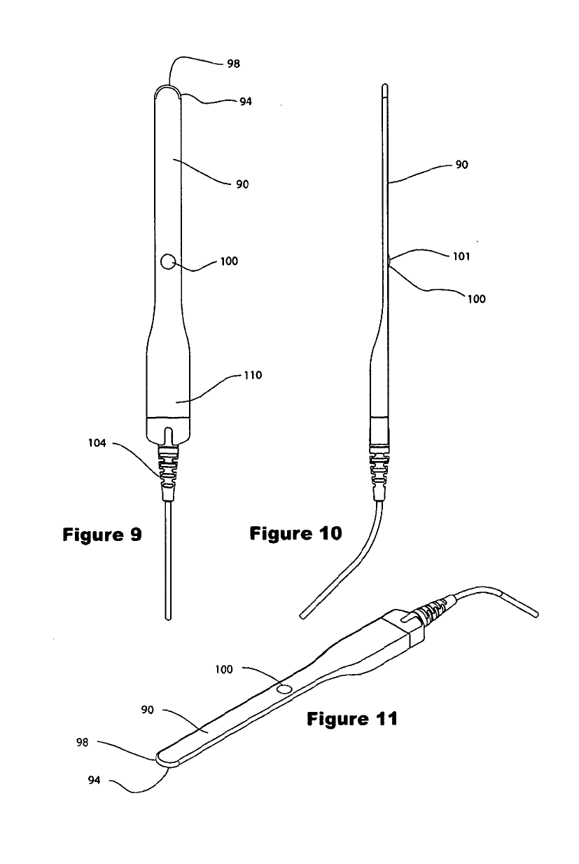

FIG. 9 illustrates an alternative design wherein an elongated shaft 90 has a distal sensor surface 94 adjacent the distal tip 98 of shaft 90 for insertion into the sublingual pocket of a patient. A proximal sensor surface 100 is mounted along the shaft 90 in a position such that the proximal sensor surface 100 is proximal the lips of the patient when the distal sensor surface 94 is positioned within the sublingual pocket. In one example for adult use, the sensor surface 100 is positioned 5 cm from the tip and may extend or be positioned along a range of 3-12 cm from the tip. The position can be shortened for pediatric use or the sensor surface 100 may be elongated (or multiple sensors deployed along the shaft) to accommodate a range of positions. In this embodiment the shaft 90 contains a distal connector 104 (such as a wire or tube) connected to one or more sensor surfaces in or on shaft 90. The shaft 90 is connected to a hub 110 which is sized to be held by the fingers of a user. The distal connector 104 extends from the hub 100 to connect to a body temperature processor (not shown) and a respiration processor (not shown) which can be combined with or part of a main processor (not shown) in a separate casing (not shown).

FIG. 10 shows a bulge 101 of sensor 100 outwardly from the surface of shaft 90.

FIG. 11 in combination with views in FIGS. 9 and 10 illustrates that shaft 90 is an elongate rectangular shape. This shape is conducive to establishing directional placement of shaft 90 in the oral cavity of a patient such that sensor surface 100 is oriented towards the external orifices of the nasal cavity (not shown). Distal tip 98 is rounded in order to ensure comfort when nested in the sublingual pocket as well as to provide a broad contact surface for sensor surface 94 to tissue in the sublingual pocket. Shaft 90 may be rectangular, square or any other combination of rectilinear cross sectional shapes conducive to establishing one or more generally flat surfaces.

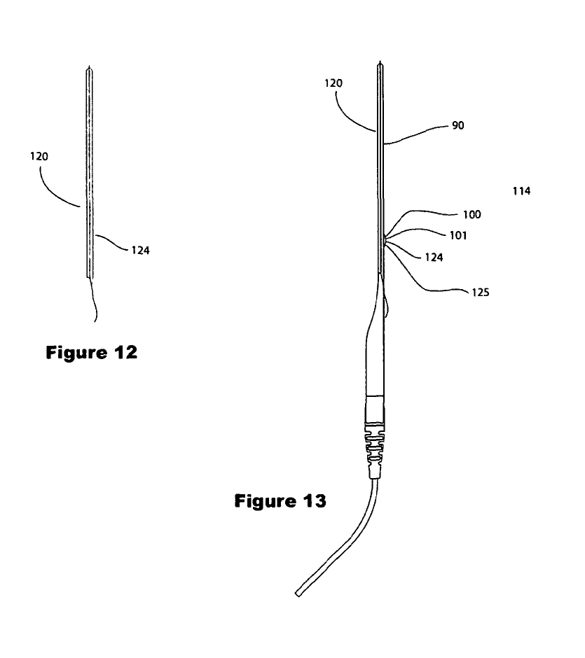

FIG. 12 discloses a flexible plastic cover 120 which is provided for use in covering all or portions of an elongate shaft. Cover 120 can be comprised of a plastic which readily transmits heat energy as is known in the art. Cover 120 can be of uniform thickness or have a thin region 124 for positioning adjacent a proximal sensor surface (not shown) which can be stretched by the sensor surface so that heat energy is more rapidly transmitted to the sensor surface. Further, cover 120 can have a shape or profile substantially equal to the shape or profile of the shaft it is covering such that sliding placement of cover 120 on a shaft (not shown) will be substantially uninhibited except for any surfaces or features of the shaft which are generally anomalous to the overall contour of the shaft which will induce deformation of cover 120 resulting in stretching of cover 120 local to that feature.

FIG. 13 shows cover 120 in place over shaft 90 with region 124 being stretched by bulge 101 of sensor surface 100 such that contact 125 is made between cover 120 and sensor surface 100 for rapid and effective transmission of heat energy across one or more material layers.

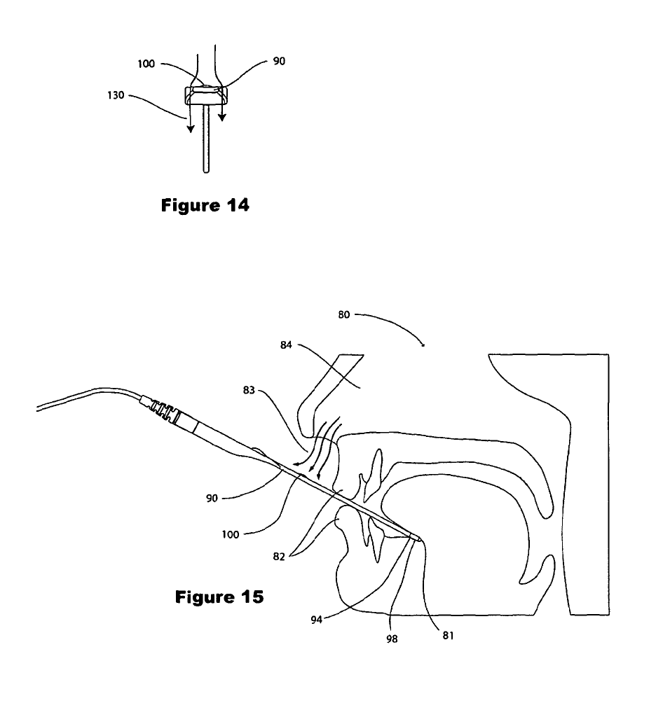

FIG. 14 shows the rectangular nature of shaft 90, with sensor surface 100 contained on a surface thereof and the directional flow of gases 130 onto and across shaft 90 and sensor surface 100. In this configuration the rectangular and generally flat shaft 90 with sensor surface 100 on one side promotes placement of shaft 90 such that sensor surface 100 is oriented towards the external orifices of the nasal cavity (not shown). Further, the generally flat nature of shaft 90 hinders the passage of exhaled gases 130 such that the gases linger in the area of sensor surface 100 longer allowing the discreet sensor or cluster of sensors (not shown) beneath sensor surface 100 to have greater exposure to the heat of the exhaled gases 130 with enhanced sensitivity to thermal variation of cyclical exhalation.

FIG. 15 is a cutaway view of the oral and nasal cavity 80 of a human being with shaft 90 shown in place. Sensor surface 94 of distal tip 98 is shown in the sublingual pocket 81. The lips 82 are shown pressed to shaft 90 in order to affect a seal on shaft 90 ensuring exhaled gas 83 flows from the nasal cavity 84 onto and across sensor surface 100.

The processor 48 is programmed to generate a waveform of tidal breathing, to count the tidal breaths and to quantify the amplitude of the tidal breaths and to output the waveform, the respiratory rate and an index or measure of the tidal amplitude, slopes, I:E ratio, and other parameters, as for example described in U.S. patent application Ser. Nos. 11/351,449, and 11/351,961 filed Feb. 10, 2006, the entire contents of each of which is incorporated by reference as if completely disclosed herein.

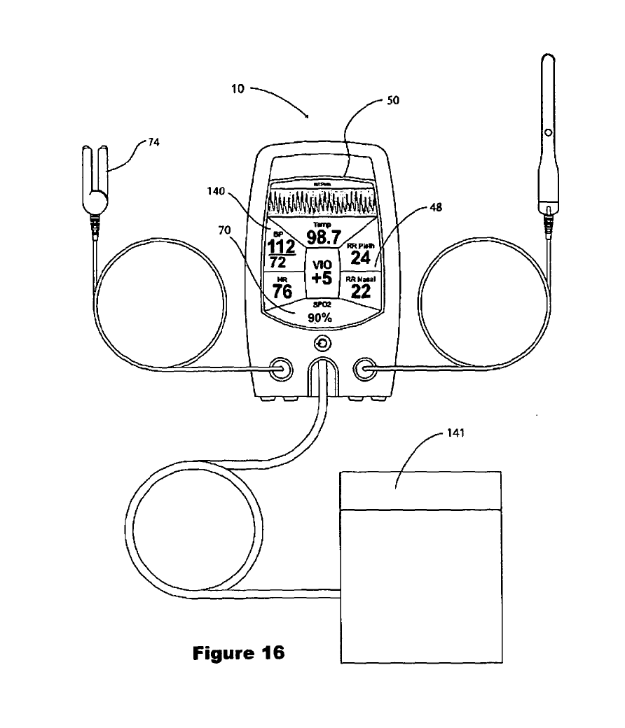

FIG. 16 shows one embodiment of the vitals monitor 10 which includes a pulse oximeter 70 with a sensor 74 for interfacing with a body part such as the finger and a blood pressure monitor 140 with a blood pressure cuff 141 for interfacing with a body part such as the upper arm. The processor 50 can be programmed to store each spot reading and to compare and output the trends of the spot reading of respiration rate from the gas flow, SP0₂, pulse, blood pressure and temperature and the derivatives of these measures, as for example described in the above patents and patent applications.

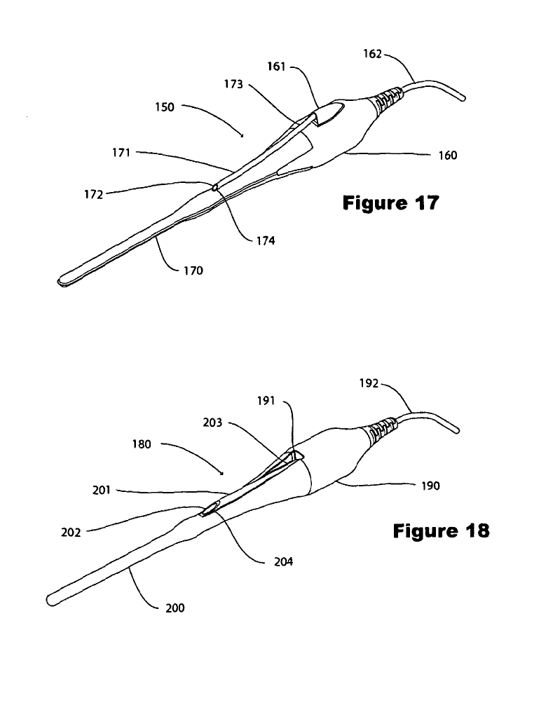

FIG. 17 describes a further embodiment of the probe 150 having a hub 160 and a disposable sheath 170. Port 161 of hub 160 has a passage there through (not shown) which is contiguous with tubing 162 that creates a continuous hollow flow path to the processor (not shown). A tube 171 is configured on sheath 170 such that it has a distal end 172 and a proximal end 173. When sheath 170 is in place on probe 150 the proximal end 173 of tube 171 is designed to be connectedly received into port 161 of hub 160 with said connection preferably being sealed or substantively sealed one to another. In this manner a continuous flow path is created between an opening 174 of tube 171 along sheath 170 and the processor (not shown) to permit continuous or intermittent sampling of exhaled gases emanating from a patient. The processor (not shown) would provide suction such that exhaled gases from the patient are drawn in through opening 174 of distal end 172 of tube 171 in order to feed said gases to the processor (not shown) in order to sample said gases and provide measures of such gases. Measured gases could be Nitric Oxide or any of the aforementioned gases or other gases or combinations thereof.

FIG. 18 describes another embodiment of the invention with probe 180 having a hub 190 and a disposable sheath 200. A common manufacturing technique with regard to such disposable sheaths is to mold them in a substantively rigid plastic manner such that the profile of the sheath is the same or nearly the same as the profile of the device they are designed to cover. When molding thermoplastics it is often necessary to do so in a way that permits design and use of inexpensive molding techniques where the molds open and close without the need for action in the tool to accommodate complex internal part geometry. It is further an important aspect of the present invention to allow sampling of exhaled gases to be done at or very close to the source of the exhaled gases. A further important aspect of the present invention is to allow for sampling of exhaled gases in a way that is not detrimental to or otherwise in interference with physical characteristics of a patient. For this reason the tube 201 of sheath 200 is configured in close proximity to the shaft of sheath 200. In this manner the opening 204 at the distal end 202 of tube 201 can sample effectively with minimal risk of occlusion. Tube 201 is substantively straight and in alignment with the main axis of sheath 200 and subsequently of probe 180. In order to facilitate such a design port 191 is recessed interiorly to hub 190 to facilitate connection of the proximal end 203 of tube 201 to an opening (not shown) in port 191 which is contiguous with tubing 192 and creates a continuous flow path from opening 204 to a processor (not shown). When sheath 200 is in place on probe 180 the proximal end 203 of tube 201 is designed to be connectedly received into port 191 of hub 190 with said connection preferably being sealed or substantively sealed one to another. In this manner a continuous flow path is created between an opening 204 of tube 201 along sheath 200 and the processor (not shown) to permit continuous or intermittent sampling of exhaled gases emanating from a patient. The processor (not shown) would provide suction such that exhaled gases from the patient are drawn in through opening 204 of distal end 202 of tube 201 in order to feed said gases to the processor (not shown) in order to sample said gases and provide measures of such gases. Measured gases could be Nitric Oxide or other gases or combinations thereof.

In an alternative embodiment plastic cover 200 can have a proximal port (not shown) in the location of a thinned region (not shown). The port can be comprised of a filter or membrane capable of transmitting C0₂ there through to a second proximal sensor (not shown) measuring the C0₂ in the exhaled gas from the nose. Alternatively the second sensor is replaced by a tube which carries the C0₂ back through hub 190 and then out to a capnometer (not shown) in a casing (not shown).

In another embodiment the casing includes an exhalation sensor (such as an infrared sensor) which can comprise a fast response infrared camera. The camera nurse points the device at the nose for example while the patient has the thermometer in the mouth with the mouth closed. The processor converts the digital detection of the infrared envelope from below the nose into a graph indicative of the heat envelope of tidal breathing and this is displayed on the screen so the nurse knows that the camera is pointed properly although a digital image in visible light can also be provided and or superimposed to assure proper positioning. The processor is programmed to count the respiration rate and other parameters as discussed. This embodiment has the advantage of avoiding a proximal heat sensor in the thermometer but is more expensive than the aforementioned embodiments.

Embodiments may include a medical device including a sensor, a microprocessor, memory, and a display. The sensor may be configured to produce a signal including a sequence of numerical values for a physiological parameter related to respiration and or temperature which may be measured over a time period and the microprocessor may be configured to process the signal. The display may present a graphical output of the ventilation expiration flow-time curve, for example, to allow manual or automatic confirmation of proper placement. The memory may be configured for storing programs and the contents of the memory may include machine readable instructions configured to direct the microprocessor to obtain the signal from the sensor and calculate a value and an index from a combination of signals. The instructions may also direct the microprocessor to calculate a trend or relational trend of the signal(s) and compare the trend or relational trend to a normal range of trends value to identify an abnormal trend.

Embodiments may include a tangible, machine readable medium that may include code which, if executed, may cause a microprocessor to obtain a signal made up of a sequence of numerical values for a physiological parameter over a time period and output a value, trend or index.

In another embodiment the relational pattern of the SPO₂, photoplethysmographic pulse, and airflow and/or other signals are analyzed to determine the presence of a pattern indicative of Cheyne Stokes Respiration and to determine the severity of Cheyne Stokes respiration especially for use in monitoring patients at home with chronic heart failure. The processor is programmed to calculate the cycling frequency and amplitude or other derivative (which may be relational) as derived from a transform such as FFT or wavelet transformation of the time series and/or time-series components or segments derived from the signals noted associated with the cycling rise and/or fall in measures of the parameters such as frequency, slope, amplitude, shape, and/or area to name a few. A few examples of these would be the cycling frequency of respiration rate, inspiration amplitude, expiration amplitude, inspiration slope, expiration slope, and cycling rise and/or fall in SPO₂ and cycling rise and/or fall in pulse frequency the slopes of the rise and fall events of the cycles along each of the respective parameters. The presence of a particular pattern of breathing such as Cheyne Stokes Respiration or of a cardiac arrhythmia such as atrial fibrillation can be displayed on the monitor.

In one embodiment accessory input for a nasal mounted thermister, nasal cannula, and/or other nasal or mask interface may be provided. This allows the device to also function for continuous monitoring (such as overnight home monitoring) if desired. The above parameters and derivatives can be combined and tracked to confirm the presence of Cheyne Stokes Respiration and to differentiate Cheyne Stokes Respiration from obstructive sleep apnea. For example, the processor can be programmed to detect the dominate cycling frequency of at least one of the parameters (as for example the frequency of the longest cluster of cycles). The processor detects the pattern of the dominate frequency over time, for example over a single night and over a period of months and/or years. If the dominate frequency demonstrates a consistent fall or rise over time the processor is programmed to detect this fall or rise pattern and output an indication which may be a warning. According to one aspect of the present invention an alarm processor may be programmed to provide an output when a threshold or pattern of fall in cycling frequency, and/or a relational image of such as fall with a rise in the ventilation oximetry index and/or increasing amplitude and/or slope of the rises and falls of the cycling.

One processing method whereby an embodiment of the present invention discriminates Cheyne Stokes Respiration from obstructive sleep apnea comprises comparing the amplitude of the falls and/or rises of the cycles to the cycle length and/or frequency. The processor is programmed to designate the pattern as indicative of Cheyne Stokes Respiration if the cycle amplitude within a given overnight study or multiple overnight studies is independent of the cycle length and to output an indication of sleep apnea if cycle length demonstrates dependency on the cycle amplitude. This can be quantified using a transform based output such as a wavelet or FFT transform wherein the degree of similarity of the cycle length can be quantified in relation to the degree of similarity of the cycle amplitude (which may, for example, be the dominate cycle frequency, and/or the cycle frequency of a measure of the clusters, (such as a majority of the clusters, or the longest cluster) such that the nature of the cycle wavelength is quantified in relation to wave amplitude and/or slope of the rise and/or fall.

While the present disclosure described above may be susceptible to various modifications and alternative forms, specific embodiments have been shown by way of example in the drawings and have been described in detail herein. The present disclosure is intended to cover all modifications, equivalents, and alternatives falling within the spirit and scope of the present disclosure.

1. A vitals monitor comprising an elongated probe having a distal tip and a disposable sheath over the probe, the monitor having a distal sensor surface adjacent the distal tip for sensing oral temperature of a patient and at least one proximal gas receiver positioned along the probe and/or sheath so that, when the distal tip is placed under a patient's tongue, exhaled gas from the nose strikes the gas receiver, the gas receiver comprising either:
a) a thin region or
b) a passage along the disposable sheath for detecting and/or measuring at least carbon dioxide in the exhaled gas,
the monitor being programmed to output at least one signal indicative of at least one ventilation parameter.

2. The vitals monitor of claim 1 wherein the gas receiver comprises a thin region along the disposable sheath and the probe has a thermal sensor for sensing nasal and/or oral airflow in contact with the thin region.

3. The vitals monitor of claim 1 wherein the gas receiver comprises a pressure sensor for sensing pressure generated by nasal and/or oral gas flow.

4. The vitals monitor of claim 1 wherein the gas receiver comprises a thin region along the sheath, the probe carrying a thermistor in contact with the thin region.

5. The vitals monitor of claim 1 wherein the gas receiver comprises a thin region along the sheath, the probe carrying a rapid response thermistor in contact with the thin region, the thin region being stretched over the thermistor.

6. The vitals monitor of claim 1 wherein the gas receiver comprises at least one of a CO₂ sensor, a fiber-optic sensor, an electromagnetic sensor, an infra-red sensor, a pressure sensor, a flow sensor, a pH sensor, a piezoelectric sensor, a chemiresistor array, PVC film, a pressure sensitive film, a heat sensitive film, a carbon nanotube sensor, and a gas collector.

7. The vitals monitor of claim 1 wherein the ventilation parameter comprises at least one component, derivative, or relationship of at least one of magnitude, frequency, slope, and/or pattern of at least one of, respiratory rate, tidal volume, inspiration, expiration, exhaled carbon dioxide, exhaled carbon monoxide, exhaled alcohol, nitric oxide, methyl nitrate, hydrogen peroxide, breath condensate, and minute ventilation.

8. The vitals monitor of claim 1 wherein the gas receiver is positioned along the elongated probe so that it is proximal to the tongue when the distal tip is positioned in the sublingual pocket so that the gas receiver can be in contact with ventilated gas moving through the mouth and/or nose.

9. The vitals monitor of claim 1 wherein the gas receiver is positioned along the probe so that it is proximal to the lips when the distal tip is positioned in the sublingual pocket so that the gas receiver can be in contact with ventilated gas moving through the nose.

10. The vitals monitor of claim 1 further comprising at least one processor for processing a parameter indicative of ventilation.

11. The vitals monitor of claim 10 wherein the processor is programmed to detect and output the frequency of tidal ventilation.

12. The vitals monitor of claim 10 wherein the processor is programmed to detect and output at least one pattern of ventilation.

13. The vitals monitor of claim 10 wherein the processor is programmed to identify and output at least one pattern of ventilation.

14. The vitals monitor of claim 10 wherein the processor is programmed to identify and output at least the pattern of Cheyne Stokes respiration.

15. The vitals monitor of claim 10 wherein the processor is programmed to identify and output at least one of the frequency and amplitude of the cycles associated with Cheyne Stokes respiration.

16. The vitals monitor of claim 10 wherein the processor is programmed to identify and output at least one pattern of heart rate.

17. The vitals monitor of claim 10 wherein the processor is programmed to identify and output at least one of the patterns of atrial fibrillation and of paroxysmal tachycardia.

18. The vitals monitor of claim 1 further comprising a pulse oximeter with at least one sensor for sensing the plethysmographic pulse and a processor for comparing the plethysmographic pulse to the parameter indicative of ventilation.

19. The vitals monitor of claim 18 wherein the processor is programmed to calculate the respiratory rate from both the plethysmographic pulse and the parameter indicative of ventilation.

20. The vitals monitor of claim 19 wherein the processor is programmed to compare the respiratory rate from the plethysmographic pulse to the respiratory rate from the parameter indicative of ventilation.

21. The vitals monitor of claim 10 wherein the processor is programmed to compare at least one parameter indicative of ventilation to at least one of the blood pressure, the pulse, the SPO₂, and the temperature and to provide an output indicative of the comparison.

22. The vitals monitor of claim 1 further comprising a transmitter configured such that the elongated probe is wirelessly connectable to an output receiver.

23. The vitals monitor of claim 1 comprising a processor programmed to compare and output the cycle length of tidal variation with the cycle amplitude of tidal variation.

24. The vitals monitor of claim 1 where the disposable sheath is configured to rapidly transmit thermal energy to the receiver.

25. The vitals monitor of claim 1 wherein the disposable sheath has at least one region which allows rapid heat transfer through the region for rapidly transmitting thermal energy to the receiver juxtaposed the region.

26. (canceled)

27. The vitals monitor of claim 1 wherein the gas receiver comprises a passage, the passage being integral with the disposable sheath.

28. The vitals monitor of claim 1 wherein the gas receiver comprises a passage, the passage being a tube for transmitting gas to at least one sensor.

29. The vitals monitor of claim 28 wherein the tube is integral with the disposable sheath.

30. The vitals monitor of claim 1 wherein the gas receiver comprises a passage, and the vitals monitor further comprises a port for connection with the passage along the disposable sheath.

31. The vitals monitor of claim 1 wherein the probe has at least one flattened portion for receiving gas from the nose.

32. The vitals monitor of claim 1 further comprising an indicator which provides an indication of the position of the probe in relation to the gas flowing from the nose.

33. The vitals monitor of claim 32 wherein the indicator comprises a display.

34. The vitals monitor of claim 32 wherein the indication comprises a graphical output.

35. The vitals monitor of claim 32 wherein the indication comprises a graphical output indicative of ventilation.

36. The vitals monitor of claim 32 wherein the indication comprises a graphical output of at least one time series of ventilation.

37. The vitals monitor of claim 32 wherein the indication comprises a graphical output of at least one flow-time curve of exhalation.

38. The vitals monitor of claim 32 wherein the indication provides for manual or automatic confirmation of proper placement of the probe.

39. Use of a vitals monitor according to claim 1 in a method of evaluating physiological parameter data including respiratory data and/or temperature.
